(19) United States
(12) Patent Application Publication
(10) Pub. No.: US 2008/0095735 A1
(43) Pub. Date: Apr. 24, 2008

(54) Skin-Protective Polymer For Use In Teat Care Compositions

(76) Inventor: Robert D. Kross, Bellmore, NY (US)

Correspondence Address: COLEMAN SUDOL SAPONE, P.C., 714 COLORADO AVENUE, BRIDGE PORT, CT, 06605-1601, US

(21) Appl. No.: 11/795,454

(22) PCT Filed: Jan. 31, 2005
(86) PCT No.: PCT/US05/02906

§ 371 Date: Jul. 17, 2007

(51) Int. Cl. A61K 31/785; A61P 17/16

(52) U.S. Cl. 424/78.07

(57) ABSTRACT

This invention relates generally to the field of antimicrobial teat care compositions. More particularly, it relates to a skin-adhering antimicrobial composition comprising a copolymer which contains methacrylic acid and 2-acrylamido-2-methyl-propane sulfonic acid (AMPS) monomeric units, the antimicrobial composition hindering the transfer of bacteria from the environment to underlying teat surfaces and orifices, and which is compatible with both mammalian tissue, including skin, and teat dip compositions.

### TECHNICAL FIELD

This invention relates generally to the field of antimicrobial teat care compositions. More particularly, it relates to a skin-adhering antimicrobial composition which hinders the transfer of bacteria from the environment to underlying teat surfaces and orifices, and which is compatible with both mammalian tissue, including skin and teat dip compositions.

### BACKGROUND OF THE INVENTION

Mammalian teat skin is particularly susceptible to damage under adverse ambient conditions, such as windy, wet, and cold weather, when chapping most often occurs. Irritated, chapped and cracked skin tissue can readily harbor pathogenic bacteria, including those that cause mammalian mastitis. Sore mammalian teats are particularly sensitive to the attachment of the milking claws, which often limits the ability of dairymen to milk the affected quarter.

Mastitis, which affects at least half of the dairy animal population to some degree, is by far the most prevalent and costly disease affecting dairy herds. Mastitis is an inflammation of the mammary gland, which can result from injury, but is principally caused by invasion of bacteria through the teat orifice during activities related to milking. It results in a lower milk output and quality, accounting for annual losses in the U.S. alone approaching $2 billion. Contagious microorganisms on contaminated equipment and hands can be transferred to the teat during the milking process, and thereafter can be transferred from cow to cow or cow to human to cow. Alternatively, environmental microorganisms which are present in the cow's surroundings, including soil, bedding, feces, and contaminated water, deposit on the teat and udder between milking periods, and can infect the udder during the time that the teat orifice remains open post-milking.

Pre- and post-milking teat dips are widely used to reduce the incidence of mastitis. Teat dips generally embody antimicrobial agents, which reduce or destroy all such pathogens, and may contain humectants, thickening and/or barrier-forming agents, and colorants. These dips are usually liquid compositions, and are most often single solutions or suspensions. In recent years, two-part teat dips have also been employed, wherein the active antimicrobial agent is formed by combination of certain chemical compounds in both parts shortly before application. The antimicrobial agents most often found in single part systems include iodophors, quaternary ammonium compounds, organic sulfonates, and chlorhexidine. Two-part systems, most often, are based on the generation of chlorous acid and/or chlorine dioxide by combination of a metal chlorite in one part and an acid source in the other.

The barrier-forming agents referenced above are incorporated into some post-milking dips so as deposit a protective film on the teat skin upon evaporation of the liquid teat dip's solvent. The protective film, in essence a “pseudo-skin,” serves to prevent or markedly reduce the penetration of bacteria to the teat skin and, in particular, the teat orifice, which can remain open for a finite period following milking. One such film former is poly(vinyl alcohol), which does not add any measurable viscosity to the teat dip composition but provides a coherent film on the teat upon solvent evaporation. Two-part teat dips, comprised primarily of acidified chlorite compositions, provide a particularly harsh environment which is inimical to the stability of barrier-forming components, generally polymeric materials, incorporated into the chlorite phase of the two-part teat dips. Kross et al., in U.S. Pat. No. 4,891,216, teach the use of a polysulfonic acid salt, specifically polymers of 2-acrylamido-2-methylpropane sulfonate (AMPS), for incorporation into acidified chlorite teat dips, to resist the degradative action of the chlorite phase into which it is initially incorporated as well as the chlorous acid composition upon combination of the two parts. The stability of the polymer is attributed to the —C—C— backbone of the polymer, which resists oxidation to a much greater degree than the —C—O—C— moieties in a wide variety of carbohydrate-based polymers.

Subsequent to that development Kross, in U.S. Pat. No. 5,507,561, taught the use of the basic polyacrylamide polymer, rather than its specific methylpropane sulfonate salt, as a component of adherent skin compositions based on acidified chlorite disinfecting formulas. All of the polyacrylamides, including the sulfonate salt, will impart some thickening to the composition, as well as forming the residual “skin.” A disadvantage of these materials is the difficulty in handling them during production. The polyacrylamides (not the sulfonate salts thereof) are generally available in powder form, and significant effort is required to incorporate them into the required solutions, which are at least 25% in concentration, as per U.S. Pat. No. 5,507,561. The sulfonate salt is most often produced in aqueous concentrations of 12%-16%, since the polymerization reaction during which the form requires reaction-sustaining concentrations at that level in order to proceed. It is often difficult to find industrial supplies and suppliers of these sulfonate solutions, and companies involved in the production of teat care formulations are generally loathe to deal with sole-source suppliers of necessary ingredients.

This patent is the result of a search for other, more readily available skin-forming polymers, for use in so-called barrier teat dips, that would be resistant to oxidizing environments and provide suitable compatibility with sensitive teat tissues, including that in the streak canal orifice.

### OBJECTS OF THE INVENTION

Accordingly, it is a general object of the present invention to supplement the limited number of skin-forming polymer barrier materials that are available for use in mammalian teat dips.

Another object of the present invention is to provide a polymeric barrier material that is resistant to the action of oxidizing antimicrobial agents used in teat dip compositions.

A further object of the present invention is to provide a viscous thickening agent that is non-irritating to mammalian tissue, and suitable for reducing drippage of a teat dip composition.

A still further object of the present invention is to provide a polymeric, barrier-forming agent for use in a teat dip such that the resulting, dry teat dip film is readily removable with water immediately prior to subsequent milking.

A more specific object of the present invention is to provide a viscous topical gel composition that will form a long-lasting solid shield on mammalian teats for the prevention of mastitis.

Still an additional object of the invention is to be provide methods for disinfecting or inhibiting the growth of bacteria on mammalian tissue, including skin surfaces, especially mammalian teat surfaces.

Any one or more of these and/or other objects and advantages of the present invention will become apparent from the following summary of the invention and description of the preferred embodiments.

### BRIEF DESCRIPTION OF THE INVENTION

The present invention provides an aqueous composition for disinfecting mammalian teat substrates that comprises a polymeric thickener, which causes the composition to adhere to the substrate to form a protective skin-like barrier. The disinfecting composition may comprise virtually any antimicrobial in current use, or yet to be developed, where a higher viscosity is desired and where a tissue-compatible protective dried film is created upon evaporation of the volatile aqueous-based solvent. The thickening, barrier-forming component of the disinfecting composition is a water-soluble copolymer of methacrylic acid and acrylamidomethyl propane sulfonic acid, used at a concentration of about 0.25% to about 5% by weight of the disinfecting composition.

Thus, preferred aspects of the present invention are directed to a topical antimicrobial composition comprising water, an effective amount of a germicidal agent and an amount of a copolymer formed from a polymerization reaction mixture comprising methacrylic acid and 2-acrylamido-2-methylpropane sulfonic acid (AMPS) monomers, said copolymer being included in said composition in an amount effective to produce a barrier when said composition is applied to mammalian teat tissue, said composition optionally comprising at least one additive selected from the group consisting of surface-active agents, pH modifiers, buffers, colorants, opacifiers, a secondary thickener, a non-thickening film former, a rheology modifier and mixtures thereof.

In preferred aspects of the present invention, the present antimicrobial compositions comprising a germicidal agent selected from the group consisting of iodophors, quaternary ammonium compounds, organic sulfonates, chlorhexidine, chlorous acid, chlorine dioxide, nitrous acid germicides and mixtures thereof. Examples of teat dips containing such materials may be found in U.S. Pat. Nos. 5,618,841, 5,651,977, 5,720,984, 6,544,539, 4,945,110 and 4,049,830 as well as U.S. Patent Appl. No. 20040180015, relevant portions of which are referenced herein.

In the composition according to the present invention, preferred copolymers comprise methacrylic acid monomers at a relative mole % ratio in said reaction mixture (or in said copolymer) which is in the range of about 60-80 moles % and AMPS monomer at a relative mole % ratio in said reaction mixture (or in said copolymer) which is in the range of about 20-40 mole %. The reaction mixture generally comprises at least about 80%, at least about 90%, at least about 95%, at least about 99% and at least about 99.9-100% by weight of methacrylic acid and AMPS monomers, with the weight % of the methacrylic acid being at least about 1.5 times and preferably at least about 2 times the weight % and up to about 9-10 times the weight % of the AMPS monomer in the reaction mixture. In certain preferred aspects of the present invention, the monomers in the reaction mixture which produces the copolymer exist as their sodium salts in the reaction mixture. The preferred copolymer which is used in compositions according to the present invention has a mean molecular weight Mₙ in the range of about 10⁴ and preferred compositions are colorless, odorless and easy to handle in the production of teat care compositions. The compositions according to the present invention are preferably adapted for use as a teat dip composition and are resistant to oxidative acidic or alkaline degradation from other components within the composition. Accordingly, preferred compositions are compatible with mammalian skin tissue, especially teat tissue.

The compositions according to the present invention have increased viscosity of at least 100 cps (centipoise units) or more, alternatively at least about 500 cps, at least about 1000 cps, at least about 2000 cps, at least about 5000 cps, at least about 10000 cps or more, the composition in its preferred form being adapted to form a protective film on mammalian skin, especially a mammalian teat to which said composition is applied soon after application and drying. In preferred aspects according to the present invention, the composition forms a film upon drying which is readily removable with water. Antimicrobial compositions according to the present invention preferably comprise copolymer at levels ranging from about 0.25% to about 5.0% by weight of said composition.

### DETAILED DESCRIPTION OF THE INVENTION

This invention teaches the use of a family of polymers that possess desirable properties for incorporation into teat dips. Its qualities recommend it for use in teat dip compositions incorporating a wide range of antimicrobial materials, including both non-oxidizing and oxidizing disinfecting agents. It is particularly suited for use in oxidizing teat dip compositions, because of its resistance to oxidative degradation. The material comprises the family of copolymers of methacrylic acid and 2-acrylamido-2-methylpropane sulfonic acid [AMPS].
The structure of the methacrylic acid monomer is 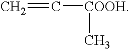
That for the AMPS monomer is

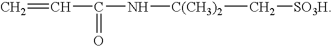

The polymerization reaction may be accomplished by solution, emulsion or suspension polymerization, which is a random polymerization. The medium for the polymerization is conveniently water, an alcohol or a mixture thereof. The choice of the medium is best dictated by the requirements of the final composition to be formulated. Although the specific details of the polymerization procedures used by the manufacturers of these types of products are generally proprietary to the organizations, it can be generally stated that the polymerization reaction is temperature, pH and catalyst sensitive. It is also desirable to exclude oxygen from the reaction vessel used to form the polymer, as this material inhibits the polymerization process. Catalysts, which are included to enhance the rate of polymerization, are materials such as ammonium bisulfite, ferrous sulfate, hydrogen peroxide, sodium metabisulfite or other redox catalysts. In general, too rapid polymerization results in lower molecular weight polymers, so usage levels and the nature of the catalyst should be carefully selected. Also playing a role are pH and the rate of addition of monomers to the reaction vessel. The use of the sodium salts of these monomers facilitates their reaction.

One commercial product composed of these copolymerized monomers is Ondeo-Nalco's Fixomer A-30, consisting of 70 mole % of the methacrylic acid and 30 mole % of the AMPS moieties. Another is their Fixomer N-28, with a 72/28 mole % ratio respectively. These products are aqueous solutions of the sodium salts of the two copolymerized moieties, containing approximately 16% total solids. Both polymers have average molecular weights Mₙ in the range of about 10⁴ and the solutions are colorless, odorless and are easy to handle in manufacturing, owing to their complete solubility in water. The viscosities are within a workable range for manufacturing; e.g., 14,500 cps at 25° C. for the Fixomer A-30, with a marked reduction in viscosity upon heating. The polymers are compatible with anionic, nonionic and amphoteric surfactants as well as other thickening agents included in teat care formulations.

Experience to date with an acidified nitrite teat care product has shown that a level of Fixomer A-30 of 1.1% contributes excellent skin barrier protection, with full stability and compatibility in the presence of other standard teat dip components. Other use levels can be established, based on the desired qualities of the teat dip and, of course, the nature of the germicidal agent. The use range of the two methacrylic acid/AMPS copolymer materials cited above, on the commercial product basis, is about 0.25% to about 5%, in the topically-applied product basis. The use level of other copolymer solutions of this type may vary, based on the level of solids in the aqueous solution and the mole ratio of the two components, such that the appropriate level may be different in order to achieve the preferred viscosity and the intended physical qualities of the dry film formed therefrom, when used in the corresponding teat dip.

The antimicrobial, barrier teat dip can be produced with any of the large number of recognized germicidal agents in current use in the veterinary field. This includes, but is not limited to iodine-based dips, including the iodophors, as well as antimicrobial quaternary ammonium compounds, organic sulfonates, chlorhexidine, chlorous acid and/or chlorine dioxide and the recently developed nitrous acid germicides. The level of use of each of these, or combinations thereof, are well known to developers of topical antimicrobial compositions. Other standard ingredients, one or more of which are generally included in these disinfecting aqueous formulations, include surface-active agents, pH modifiers and/or buffers, colorants, opacifiers, other thickeners, non-thickening film formers, rheology modifiers and mixtures thereof, all of which are included in amounts which are effective for the role for which the component has been added to the present compositions.

It is often advantageous to generate a disinfecting composition that dries rapidly. Drying time of the disinfecting composition is important in generally, but especially when the surface will be exposed to foreign material which may stick to the adherent matrix, or for cosmetic reasons. When altering this characteristic, temperature, which tends to enhance evaporation, and humidity, which tends to suppress evaporation and increase drying time, should be taken into account. The barrier which is produced from the application and drying of the composition on the teat skin surface, especially a teat surface, should be readily removed with water.

The antimicrobial compositions according to the present invention is preferably presented as a single (single phase) composition prior to application to a skin surface. However, the composition may be stored or presented as a two part composition which is mixed prior to application When the disinfecting composition of this invention is provided in two phases, the protic acid solution or gel and the metal chlorite solution or gel are mixed in suitable ratios to generate the chlorous acid, and the disinfecting composition is then applied to the surface to be disinfected. Preferably, the two phases are combined in approximately equal parts. More preferably, the disinfecting composition is mixed immediately prior to application.

In one aspect of the present invention, the disinfecting composition may be applied to mammalian teats. The composition may be applied by any one of several means, including dipping, from one of a series of commercially available dip cups, or spraying from a nozzle suitably adjusted to dispense a gelled formulation. Although the effective amount may vary, about 0.5 to 2.0 grams of disinfecting composition is sufficient, more required if the surface is large. A more viscous formulation (ca. 1000 cps) will generally deposit closer to 2.0 grams per teat, even more viscous a greater amount.

The present invention is illustrated by the following examples. Unless otherwise noted, all parts and percentages in the examples as well as the specification and claims are by weight.

### EXAMPLE 1

This Example illustrates the use of the present invention as a barrier-forming thickening agent in a two-part teat dip for application to cow teats. A first gel is prepared by mixing the following ingredients:

| | |
|---|---|
| Fixomer A-30 copolymer (12.1% aqueous solution) | 2.24% |
| NaOH, 10 N | 0.09% |
| Nacconol 90F (sodium dodecylbenzene sulfonate) | 2.00% |
| Sodium chlorite (pure basis) | 0.64% |
| Xanthan gum (Keltrol T) | 0.50% |
| Water | q.s. |

A second gel is prepared by mixing the following ingredients:

| | |
|---|---|
| Lactic acid (pure basis) | 2.64% |
| Natrosol 250 MBR | 1.00% |
| Polyethylene glycol 400 | 1.50% |
| Sodium benzoate | 0.10% |
| Carbitol | 5.00% |
| FD&C Yellow #5 | 0.30% |
| Water | q.s. |

The two solutions are blended, preferably just prior to application. The resulting gel is applied to the cow's teat, forming a protective antimicrobial shield around the teat, which solidifies upon drying to a non-tacky surface to which environmental substances do not adhere. The film shield thus formed provides a long lasting and continuously acting disinfectant in direct contact with the skin surface.

### EXAMPLE 2

This Example illustrates the use of the present invention as a barrier-forming thickening agent in a teat dip for application to cow teats, where each of two parts of the dip is separately prepared, and where the subsequent 1:1 combination of these parts produces a stable one-part teat dip. A first gel is prepared by mixing the following ingredients:

| | |
|---|---|
| Fixomer N-28 copolymer (16.0% aqueous solution) | 2.15% |
| Xanthan gum (Keltrol T) | 0.50% |
| Dodecylbenzene Sulfonate, Na | 2.00% |
| Sodium benzoate | 0.20% |
| Sodium nitrite (pure basis) | 0.63% |
| FD % C Yellow #5 | 0.20% |
| Water | q.s. |

A second gel is prepared by mixing the following ingredients:

| | |
|---|---|
| Lactic acid (88% tech.) | 3.23% |
| Carbitol | 5.00% |
| Glycerin | 5.00% |
| Natrosol 250 MBR | 0.50% |
| Sodium benzoate | 0.05% |
| Hydrochloric acid (11.7 N conc.) | 0.43% |
| Benzalkonium chloride (17% aq.) | 1.26% |
| Water | q.s. |

The two gels are blended in a 1:1 combination and the resulting mixture is applied to the cow teat by a standard dipping procedure. This application can be made either shortly after combination of the two parts, or up to at least several weeks after mixing, forming a protective antimicrobial shield around the teat. The film solidifies upon drying to a non-tacky surface, to which environmental substances do not adhere. The film shield thus formed provides a long lasting and continuously acting disinfectant in direct contact with the skin surface.

The following study demonstrates the antimicrobial effectiveness of this composition. In this study, in vitro microbiological evaluations were run on the composition at five times; when freshly mixed (i.e., 10-minutes after combination) as well as 1, 2, 6 and 14 days after preparation.

Procedure:

The initial inoculum at each test period was >10⁸, as will be seen in the test data. The microorganism was plated on Trypticase Soy Agar and incubated at 35°-37° C. for 24 hours. A heavy suspension was prepared in sterile saline. Equal quantities (by weight) of the teat dip components were mixed together, and allowed to stand for about 10 minutes. Then nine volumes of this sample were challenged with one volume of the organism suspension for 60 seconds. Thereafter 2.0 ml of the mixture were added to 18 ml of D/E broth. A further 1/10 dilution of the D/E broth in saline was prepared. Five 2.0 ml samples of the D/E broth were added to petri plates. Duplicate 1.0 ml samples were added to petri plates, and duplicate 1.0 ml samples of the 1/10 dilution were added to petri plates. Approximately 10 ml of liquid Trypticase Soy Agar were added to each petri plate and allowed to solidify. Plates were incubated at 35°-37° C. for 2448 hours, and colony forming units were counted. Thereafter the mixed sample was incubated in a foil-covered sterile container at room temperature, until use. After the first sample (Day 0) sample was tested, samples were removed for testing 1 and 2 days after mixing (Day 1 and 2, resp.) and were tested as above. At each test point a control study was run, in which a sample of saline was challenged, instead of the test sample.

Results:

| | | | |
|---|---|---|---|
| | Challenge | | |
| | Inoculum | | |
| | (Log #cfu/ml | Organisms Recovered | Log |
| Test Sample | Product) | (Log #cfu/ml Product | Reduction |

Day 0

| | | | |
|---|---|---|---|
| Teat Dip | 2.2 × 10⁸ (8.34) | 0 | >8.34 |
| Control (Saline) | 2.2 × 10⁸ (8.34) | 2.5 × 10⁸ | |

Day 1

| | | | |
|---|---|---|---|
| Teat Dip | 4.0 × 10⁸ (8.60) | 1.7 × 10¹ (1.23) | 7.37 |
| Control (Saline) | 4.0 × 10⁸ (8.60) | 2.7 × 10⁸ | |

Day 2

| | | | |
|---|---|---|---|
| Teat Dip | 3.4 × 10⁸ (8.53) | 0 | >8.53 |
| Control (Saline) | 3.4 × 10⁸ (8.53) | 2.4 × 10⁸ | |

Day 6

| | | | |
|---|---|---|---|
| Teat Dip | 3.2 × 10⁸ (8.51) | 0 | >8.51 |
| Control (Saline) | 3.2 × 10⁸ (8.51) | 4.1 × 10⁸ | |

Day 14

| | | | |
|---|---|---|---|
| Teat Dip | 7.8 × 10⁸ (8.89) | 0 | >8.89 |
| Control (Saline) | 7.8 × 10⁸ (8.89) | 3.8 × 10⁸ | |

These results clearly demonstrate that the this nitrous acid barrier teat dip comprising the inventive copolymer was capable of destroying 220-780 million E. coli organisms within 60 seconds of contact, up through two weeks following mixture.

The principles, preferred embodiments and modes of operation of the invention have been described in the foregoing specification. The invention, which is intended to be protected herein, however, is not to be construed as limited to the particular form disclosed, since these are to be regarded as illustrative rather than restrictive. Variations and changes may be made by those skilled in the art without departing from the spirit of the invention.

1. A topical antimicrobial composition comprising water, an effective amount of a germicidal agent and an amount of a copolymer formed from a polymerization reaction mixture comprising methacrylic acid and 2-acrylamido-2-methylpropane sulfonic acid (AMPS) monomers, said copolymer being included in said composition in an amount effective to produce a barrier when said composition is applied to mammalian skin, said composition optionally comprising at least one additive selected from the group consisting of surface-active agents, pH modifiers, buffers, colorants, opacifiers, a secondary thickener, a non-thickening film former, a rheology modifier and mixtures thereof.

2. The composition according to claim 1 wherein said germicidal agent is selected from the group consisting of iodophors, quaternary ammonium compounds, organic sulfonates, chlorhexidine, chlorous acid, chlorine dioxide, nitrous acid germicides and mixtures thereof.

3. The composition according to claim 1 wherein the relative mole % ratio of the methacrylic acid monomer in said reaction mixture is in the range of about 60-80 moles % and the relative mole % ratio of the AMPS monomer is in the range of about 20-40 mole %.

4. The composition according to claim 1 wherein said monomers in said reaction mixture exist as their sodium salts in the reaction mixture.

5. The composition according to claim 1 wherein said copolymer has a mean molecular weight Mₙ in the range of about 10⁴ and wherein said composition is colorless, odorless and easy to handle in the production of teat care compositions.

6. The composition according to claim 1 adapted for use as a teat dip composition wherein said copolymer is resistant to oxidative, acidic or alkaline degradation.

7. The composition according to claim 1 wherein said copolymer is compatible with mammalian teat tissue.

8. The composition according to claim 6 having a viscosity of at least about 500 cps, said composition forming a protective film on a teat to which said composition is applied soon after application and drying.

9. The composition according to claim 6 having a viscosity of at least about 1000 cps, said composition forming a protective film on a teat to which said composition is applied soon after application and drying.

10. The composition according to claim 1 wherein said film formed upon drying is readily removable with water.

11. The composition according to claim 1 wherein said copolymer is present at levels of about 0.25% to about 5.0% by weight of said composition.

12. A method of disinfecting a mammalian skin surface, said method comprising exposing said surface to an antimicrobial composition comprising water, an effective amount of a germicidal agent and an amount of a copolymer formed from a polymerization reaction mixture comprising methacrylic acid and 2-acrylamido-2-methylpropane sulfonic acid (AMPS) monomers, said copolymer being included in said composition in an amount effective to produce a barrier when said composition is applied to said surface, said composition optionally comprising at least one additive selected from the group consisting of surface-active agents, pH modifiers, buffers, colorants, opacifiers, a secondary thickener, a non-thickening film former, a rheology modifier and mixtures thereof.

13. The method according to claim 12 wherein said germicidal agent is selected from the group consisting of iodophors, quaternary ammonium compounds, organic sulfonates, chlorhexidine, chlorous acid, chlorine dioxide, nitrous acid germicides and mixtures thereof.

14. The method according to claim 12 wherein the relative mole % ratio of the methacrylic acid monomer in said reaction mixture is in the range of about 60-80 moles % and the relative mole % ratio of the AMPS monomer is in the range of about 20-40 mole %.

15. The method according to claim 12 wherein said monomers in said reaction mixture exist as their sodium salts in the reaction mixture.

16. The method according to claim 12 wherein said copolymer has a mean molecular weight Mₙ in the range of about 10⁴ and wherein said composition is colorless, odorless and easy to handle in the production of teat care compositions.

17. The method according to claim 12 wherein said composition is adapted for use as a teat dip composition wherein said copolymer is resistant to oxidative, acidic or alkaline degradation.

18. The method according to claim 12 wherein said copolymer is compatible with mammalian teat tissue.

19. The method according to claim 12 wherein said composition has a viscosity of at least about 500 cps, said composition forming a protective film on a teat to which said composition is applied soon after application and drying.

20. The method according to claim 19 wherein said film formed upon drying is readily removable with water.

21. The method according to claim 12 wherein said copolymer is present at levels of about 0.25% to about 5.0% by weight of said composition.
